(19) United States
(12) Patent Application Publication
(10) Pub. No.: US 2020/0375459 A1
(43) Pub. Date: Dec. 3, 2020

(54) IMPLANTABLE RECHARGEABLE TELEMETRY DEVICE

(71) Applicant: NewPace Ltd., Caesarea (IL)

(72) Inventors: Gera Strommer, Haifa (IL); Avraham Broder, Petach Tikva (IL); Robert Fishel, Delray Beach, FL (US)

(21) Appl. No.: 16/882,647

(22) Filed: May 25, 2020

Related U.S. Application Data
(60) Provisional application No. 62/856,254, filed on Jun. 3, 2019.

(51) Int. Cl. A61B 5/00; A61B 5/0464; A61B 5/046; A61B 5/1455

(52) U.S. Cl. CPC A61B 5/0031; A61B 5/0464; A61B 5/1455; A61B 5/0006; A61B 5/4818; A61B 5/046

(57) ABSTRACT

An implantable rechargeable telemetry device (IRTD) for injection into a subject comprising: an ECG monitor in electrical communication with an electrode and adapted for collecting ECG data of a subject, a rechargeable battery adapted for powering the IRTD; and a wireless charging receiver adapted to receive charging current from a charging device to recharge the battery, wherein the collected ECG data is continuously monitored ECG data and wherein the IRTD further comprises a wireless communication device adapted for continuous transmission of the collected continuously monitored ECG data to an external computing device.

### CROSS REFERENCE TO RELATED APPLICATIONS

This application claims priority from U.S. Provisional Patent Application No. 62/856,254 filed Jun. 3, 2019, which is expressly incorporated herein by reference in its entirety.

### FIELD

Embodiments disclosed herein relate to systems and methods for implantable medical devices intended for collecting a patient's electrocardiography (ECG) data (or “signals”).

### BACKGROUND

Implantable ECG recorders are small implantable medical devices intended for collecting a patient's ECG data and transmitting it to a center, where such ECG data is analyzed. The ECG recorder devices are inserted under the skin of a subject in a simple “injection like” procedure in which a tiny incision is made on the skin, and a tunneling tool (usually containing the ECG recorder is used for creating space under the skin and implanting the ECG recorder.

A typical known art ECG recorder is shown in FIGS. 1A-1B. As shown in FIG. 1A, an ECG recorder 20 is implanted under the skin of a patient 10 and communicates via a communication link 22 with a portable device 40. As shown in FIG. 1B, ECG recorder 20 comprises a hermetically sealed elongated case 24 formed from a biocompatible metal such as titanium. ECG recorder 20 comprises a battery 26 for powering ECG recorder 20. ECG recorder 20 further comprises an ECG monitor 28 connected to at least two electrodes 32 mounted on non-metallic electrode mounts 30 on either side of ECG recorder 20 for collecting of at least one ECG vector. A processor 34 including storage analyses the ECG signals using software and algorithms for detecting various arrhythmias and for storing such events. The stored data can be periodically transmitted using a communication module 36 and wireless technology such as 430 MHZ RF or Bluetooth to external device 40.

In many situations it is be advantageous for a medical expert to receive constant ECG information (“telemetry”) from a patient. Continuous telemetry implies continuous transmission of ECG signals from a monitoring device such that these ECG signals may be inspected by an expert in real time or near real time. Such continuous telemetry is provided by wearable ECG devices and patches that use an external, replaceable battery, or a rechargeable battery having a direct input electrical socket for recharging. In ECG recorder 20, battery 26 typically has a limited lifetime of 2-4 years. Due to this limitation of battery 26, ECG recorder 20 cannot provide continuous telemetry as this would run battery 26 down in a very short period due to energy consumed by the continuous transmission, requiring replacement of ECG recorder 20. Hence current ECG recorder devices only enable intermittent non-real-time transmission of ECG data in short data packages.

### SUMMARY

Exemplary embodiments disclosed herein relate to an implantable Rechargeable Telemetry Device (IRTD) and method of use. The IRTD as disclosed herein is able to transmit continuous ECG telemetry, since the internal rechargeable battery working in conjunction with a wireless charging mechanism embedded as part of the IRTD may be continuously recharged. The rechargeable IRTD can be charged periodically or continually during continuous telemetry so as not to lose energy due to continuous transmission. A rechargeable battery and wireless charging mechanism also enables extension of the lifetime of the IRTD without the need for replacement. Multiple embodiments for wireless charging of the IRTD are contemplated herein and described further below.

Unless otherwise defined, all technical and scientific terms used herein have the same meaning as commonly understood by one of ordinary skill in the art. The materials, methods, and examples provided herein are illustrative only and not intended to be limiting.

In some embodiments an IRTD for injection into a subject comprises: an ECG monitor in electrical communication with an electrode and adapted for collecting ECG data of a subject; a rechargeable battery adapted for powering the IRTD; and a wireless charging receiver adapted to receive charging current from a charging device to recharge the battery. In some embodiments, the collected ECG data is continuously monitored ECG data. In some embodiments, the IRTD further comprises a wireless communication device adapted for continuous transmission of the collected continuously monitored ECG data to an external computing device.

In some embodiments, the continuous transmission takes place during recharging of the rechargeable battery. In some embodiments, the continuous transmission is activated by one of recharging the rechargeable battery or activation via the external device. In some embodiments, the recharging device is one of a wireless charging patch comprising a patch battery or a charging pad. In some embodiments, the patch battery is a rechargeable patch battery. In some embodiments, the IRTD further comprises a controller. In some embodiments, the IRTD further comprises an accelerometer and the controller is adapted for simultaneous collection of ECG data and data related to movement of a subject as tracked by the accelerometer. In some embodiments, collected accelerometer data and ECG data are used for monitoring of syncope of a subject.

In some embodiments, the IRTD further comprises a microphone and the controller is adapted for simultaneous collection of ECG data and audio data from a subject. In some embodiments, collected audio data and ECG data are used for monitoring of sleep apnea of a subject. In some embodiments, the IRTD further comprises an oximeter and the controller is adapted for simultaneous collection of ECG data and oximeter data from a subject.

In some embodiments, the charging coil is positioned on a non-metallic mount. In some embodiments, the charging coil is positioned on an outer surface the IRTD electrically isolated from a metal case of the IRTD by a non-conductive base. In some embodiments, the processor is adapted for analysis of ECG signals. In some embodiments, the analysis results in generation of an alarm related to one or more of arrhythmia, ventricular fibrillation, or pulseless ventricular tachycardia (VT). In some embodiments, alarms generated by the IRTD are transmitted to the external device. In some embodiments, the external device comprises a location function and alarms generated by the IRTD are transmitted to a remote server by the external device along with the determined location of the external device.

Implementation of the method and system of the present disclosure may involve performing or completing certain selected tasks or steps manually, automatically, or a combination thereof. Moreover, according to actual instrumentation and equipment of preferred embodiments of the method and system of the present disclosure, several selected steps may be implemented by hardware (HW) or by software (SW) on any operating system of any firmware, or by a combination thereof. For example, as hardware, selected steps of the disclosure could be implemented as a chip or a circuit. As software or algorithm, selected steps of the disclosure could be implemented as a plurality of software instructions being executed by a computer using any suitable operating system. In any case, selected steps of the method and system of the disclosure could be described as being performed by a data processor, such as a computing platform for executing a plurality of instructions.

Although the present disclosure is described with regard to a “computing device”, a “computer”, or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, a smart watch or a PDA (personal digital assistant). Any two or more of such devices in communication with each other may optionally comprise a “network” or a “computer network”.

### BRIEF DESCRIPTION OF THE DRAWINGS

Aspects, embodiments and features disclosed herein will become apparent from the following detailed description when considered in conjunction with the accompanying drawings. Like elements may be marked with like numerals in different figures, where:

FIG. 1A is an exemplary schematic drawings of a known art injectable ECG recorder inserted in a patient;

FIG. 1B shows details of the injectable ECG recorder of FIG. 1A;

FIG. 2A is an exemplary schematic drawings of an implantable rechargeable telemetry device (IRTD) according to some embodiments disclosed herein;

FIG. 2B shows the IRTD of FIG. 2A inserted in a patient;

FIG. 2C shows details of a charging pad and other components of the IRTD of FIGS. 2A and 2B;

FIG. 2D shows a charging patch and other components of the IRTD of FIGS. 2A and 2B;

FIG. 2F shows a charging coil positioned on a case in the IRTD of FIGS. 2A and 2B;

FIG. 2E shows the ITRD of FIG. 2B inserted in the patient and being charged.

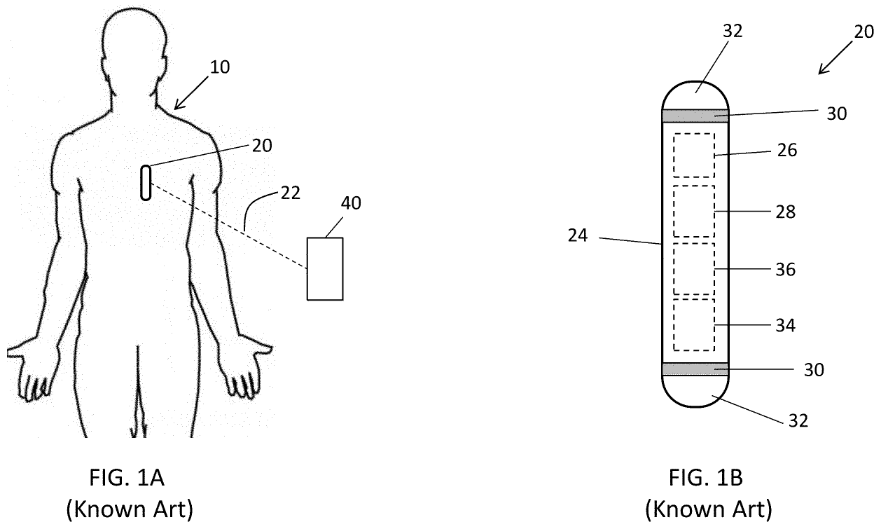

### DETAILED DESCRIPTION

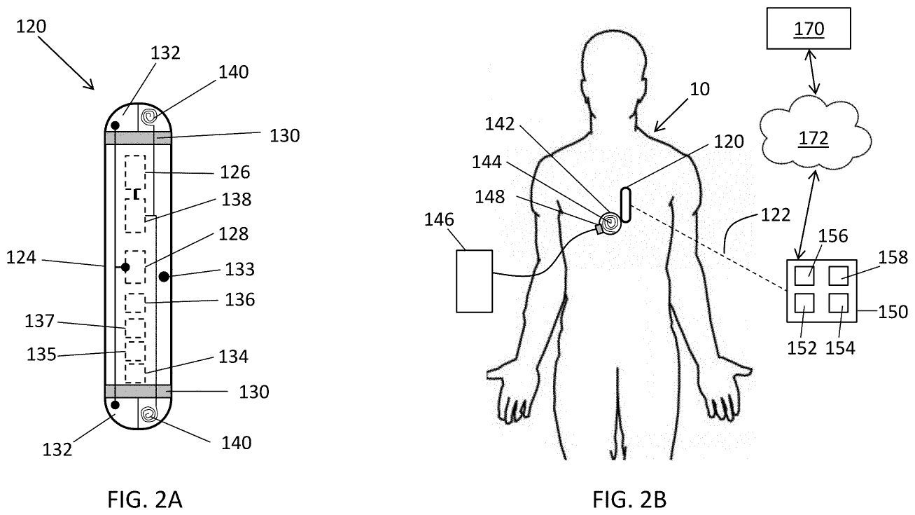

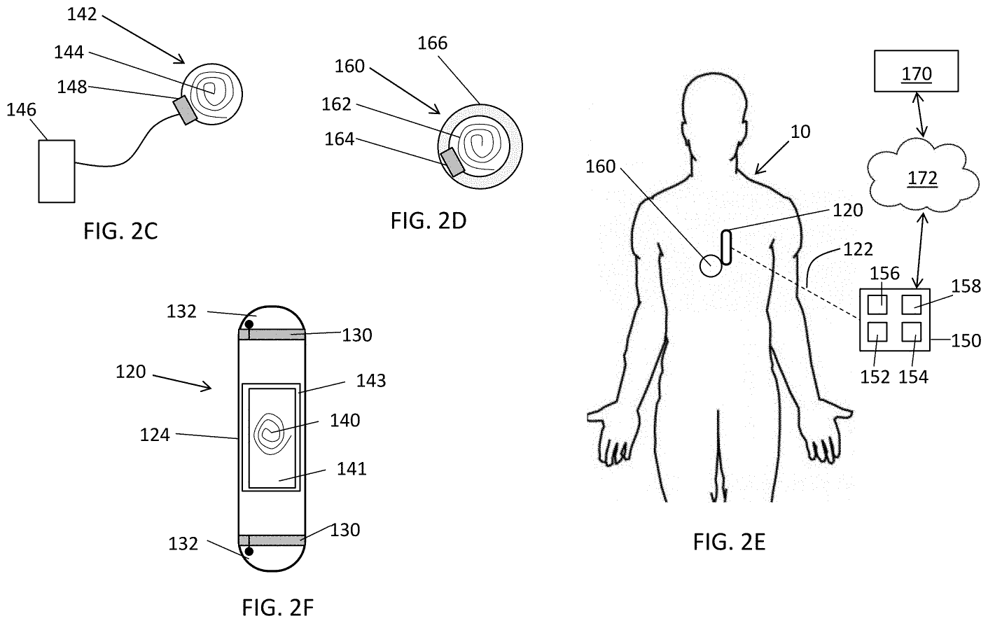

Exemplary embodiments relate to an IRTD and method of use. FIGS. 2A-2F show exemplary schematic drawings of an IRTD according to some embodiments.

As shown in FIG. 2A, IRTD 120 comprises a hermetically sealed elongated case 124 formed from a biocompatible metal such as titanium. IRTD 120 contains a rechargeable battery 126 for powering IRTD 120. Battery 126 comprises any rechargeable chemistry known in the art such as but not limited to lithium-ion. IRTD 120 also includes an IRTD charging controller 138 and at least one IRTD charging coil 140. Charging coil 140 is positioned outside of case 124 on or in non-metallic electrode mounts 130 such that the metallic case 124 does not interfere with wireless charging of IRTD 120. Alternatively, in some embodiments, as shown in FIG. 2F, charging coil 140 is positioned on an outer surface of case 124, electrically isolated from the metal case by a non-conductive base 141 and covered with a non-conductive coating 143. Charging coil 140 is attached to IRTD charging controller 138 via a suitable opening (feedthrough) in case 124.

It should be appreciated that IRTD 120 is able to transmit continuous ECG telemetry without depleting the battery, since battery 126 may be continuously recharged while simultaneously transmitting continuous ECG telemetry. Rechargeable battery 126 can be charged periodically, or during continuous telemetry so as not to lose energy due to continuous transmission.

IRTD 120 further comprises an ECG monitor 128 connected to at least two electrodes 132 mounted on non-metallic electrode mounts 130 on either side of IRTD 120 for collecting of at least one ECG vector. In some embodiments, electrodes 132 are mounted on IRTD 120 while maintaining electrical isolation from metallic pails of IRTD 120 using isolation means and/or a feedthrough. A processor 134 containing storage controls the operation of IRTD 120. Processor 134 analyses the ECG signal using software and algorithms for detecting various arrhythmias and for storing such events.

In some embodiments, IRTD 120 generates alarms such as but not limited to when ventricular fibrillation (VF) and pulseless ventricular tachycardia (VT) are determined by processor 134 to have occurred following analysis of ECG data. Alarms are provided by an audio output device (not shown) connected to controller 138, or may alternatively be transmitted for indication on external device 150.

IRTD 120 comprises a communication module 136 using a wireless communication technology such as but not limited to Bluetooth, Low Energy Bluetooth (BLE), RF 430 MHZ or any suitable technology for communication 122 with an external device 150.

In some embodiments, IRTD 120 comprises an accelerometer 135, and data related to movement of subject 10 as tracked by accelerometer 135 is collected simultaneously by IRTD 120 with the continuous ECG data. In some embodiments, accelerometer data, indicative of the movement of subject 10, is used as part of monitoring of syncope by processor 134 of IRTD 120.

In some embodiments, IRTD 120 further comprises a microphone 133, and audio data such as comments from subject 10 or sounds of subject 10 breathing are recorded along with ECG data by IRTD 120. In some embodiments, audio recording may be used along with received ECG data as part of monitoring by processor 134 in IRTD 120 of sleep apnea. In some embodiments, IRTD 120 comprises an oximeter 137 and data collected from the oximeter relating to subject 10 is recorded and optionally analyzed by processor 134 along with ECG data.

As shown in FIGS. 2B-2C, IRTD 120 is implanted under the skin of a subject 10. A charging pad 142 containing a pad charging coil 144 is positioned near implanted IRTD 120 for transmitting charging energy such as by conductive charging to IRTD charging antenna 140 of IRTD 120. Charging pad 142 receives power from external charger 146. Charging pad 146 is optionally connected to an electrical outlet or optionally contains a rechargeable battery that was previously charged. Charging pad 142 optionally comprises a pad charging controller 148 for monitoring and controlling inductive charging. Pad charging controller 148 is optionally provided as part of charger 146. In some embodiments, charger 146 is part of external device 150.

IRTD 120 is thus charged wirelessly by pad 142 using conductive charging, and may simultaneously provide continuous ECG telemetry related to subject 10. Optionally continuous ECG telemetry is not provided during charging. In some embodiments, continuous telemetry can only be activated when charging pad 142 is charging IRTD 120.

Processor 134 also monitors charging controller 138 and the charge state of battery 126 such that IRTD 120 provides an indication of the battery charge status. The indication may be an audible indication such as provided by an audio output device (not shown) connected to controller 138 or may alternatively be an indication on external device 150 or external charger 146. External device 150 may be any one of, but is not limited to, a computer, a smartphone, a smartwatch, or similar, communicating with communication module 136.

External device 150 comprises a software application (app) 152 for running on external device 150. In some embodiments, alarms generated by IRTD 120 are displayed or audibly indicated and/or transmitted to remote server 172 (see below) by app 152. In some embodiments, external device 150 comprises a location function such as a GPS receiver, and alarms generated by IRTD 120 are transmitted to remote server 172 or to an emergency service along with the determined location of external device 150 such that assistance can be dispatched to the determined location.

In some embodiments, analysis generated by IRTD 120 is displayed by app 152 and/or transmitted to remote server 172 (see below) by app 152. In some embodiments, app 152 includes a continuous ECG activation button displayed on app 152 that, when pressed, activates continuous ECG monitoring in IRTD 120, optionally enabling a choice of the time period for continuous monitoring and/or indicating that IRTD 120 must be charged during continuous monitoring.

In some embodiments, external device 150 comprises an accelerometer 154 and data related to movement of subject 10 as tracked by accelerometer 154 is collected simultaneously by app 152 with the continuous ECG data provided by IRTD 120. In some embodiments, accelerometer data, indicative of the movement of subject 10, is used as part of monitoring of syncope by app 152 and/or by remote users 170.

In some embodiments, external device 150 comprises a microphone 156, and audio data such as comments from subject 10 or sounds of subject 10 breathing are recorded using microphone 156 along with ECG data. In some embodiments, audio recording may be used along with received ECG data as part of monitoring by app 152 of sleep apnea. In some embodiments, external device 150 comprises an oximeter 158 and data collected from the oximeter relating to subject 10 is recorded and optionally analyzed along with ECG data by app 152.

In some embodiments, the ECG data transmitted from IRTD 120 to external device 150 is further transmitted by external device 150 using the built-in data communication functionality of external device 150 to remote server 170, for analysis such as by clinicians. In some embodiments, remote server 170 is one or more computing devices with appropriate software and hardware for receiving, analyzing, storing and displaying data collected by IRTD 120 and app 172. Transmission from external device 150 to remote server 170 uses any suitable wired or wireless communication protocol or network 172. In some embodiments, collected accelerometer, microphone and/or oximeter data is also transmitted along with collected ECG data from external device 150 to remote server 170.

In some embodiments, charging of IRTD 120 is performed by a wireless charging patch 160. As shown in FIGS. 2D-2E, charging patch 160 comprises a power source such as a patch battery 166. Charging patch 160 contains a patch charging coil 162 for positioning near implanted IRTD 120 for transmitting charging energy such as by conductive charging to IRTD charging coil 140 of IRTD 120. Patch battery 166 is optionally rechargeable such as from an external charger or direct connection to a power outlet. Charging patch 160 comprises a patch charging controller 164 for monitoring and controlling inductive charging of IRTD 120. In some embodiments, charging patch 160 optionally includes a sticker for being held in position over IRTD 120 for charging. In some embodiments, patch charging controller 164 comprises a wireless communication circuit using technology such as but not limited to BLE for external communication of the status and control of the charging of patch 160, such as to app 152 on external device 150. In some embodiments, patch charging controller 164 provides audible or visual indications of the charge status of patch battery 166 and IRTD battery 126.

IRTD 120 is thus charged wirelessly by patch 160 using conductive charging, and may simultaneously transmit continuous ECG telemetry related to subject 10. Optionally continuous ECG telemetry is not provided during charging. In some embodiments, continuous telemetry can only be activated when charging patch 160 is charging IRTD 120.

In some embodiments, charging patch 160 comprises an accelerometer (not shown) and data related to movement of subject 10 as tracked by the accelerometer and is communicated to app 152 on external device 150. In some embodiments, charging patch 160 comprises a microphone (not shown) and audio data such as comments from subject 10 or sounds of subject 10 breathing are recorded along with ECG data for transmission to app 152 on external device 150. In some embodiments, audio recording from patch 160 may be used as part of monitoring of sleep apnea by app 152. In some embodiments, patch 160 comprises an oximeter (not shown) and data collected from the oximeter relating to subject 10 is transmitted to app 152 on external device 150.

In the claims or specification of the present application, unless otherwise stated, adjectives such as “substantially” and “about” modifying a condition or relationship characteristic of a feature or features of an embodiment, are understood to mean that the condition or characteristic is defined to within tolerances that are acceptable for operation of the embodiment for an application for which it is intended.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

While this disclosure describes a limited number of embodiments, it will be appreciated that many variations, modifications and other applications of such embodiments may be made. The disclosure is to be understood as not limited by the specific embodiments described herein, but only by the scope of the appended claims.

What is claimed is:

1. An implantable rechargeable telemetry device (IRTD) for injection into a subject comprising:
a) an ECG monitor in electrical communication with an electrode and adapted for collecting ECG data of a subject;
b) a rechargeable battery adapted for powering the IRTD; and
c) a wireless charging receiver adapted to receive charging current from a charging device to recharge the battery.

2. The IRTD of claim 1, wherein the collected ECG data is continuously monitored ECG data.

3. The IRTD of claim 2, further comprising a wireless communication device adapted for continuous transmission of the collected continuously monitored ECG data to an external computing device.

4. The IRTD of claim 3, adapted such that the continuous transmission takes place during recharging of the rechargeable battery.

5. The IRTD of claim 2, wherein the continuous transmission is activated by recharging the rechargeable battery or by activation via the external device.

6. The IRTD of claim 4, wherein the recharging device is one of a wireless charging patch comprising a patch battery or a charging pad.

7. The IRTD of claim 6, wherein the patch battery is a rechargeable patch battery.

8. The IRTD of claim 2, further comprising a controller.

9. The IRTD of claim 8, further comprising an accelerometer and wherein the controller is adapted for simultaneous collection of ECG data and accelerometer data related to movement of a subject as tracked by the accelerometer.

10. The IRTD of claim 9, wherein the accelerometer data and ECG data are used for monitoring of syncope of a subject.

11. The IRTD of claim 8, further comprising a microphone and wherein the controller is adapted for simultaneous collection of ECG data and audio data from a subject.

12. The IRTD of claim 11, wherein the audio data and ECG data are used for monitoring of sleep apnea of a subject.

13. The IRTD of claim 8, further comprising an oximeter and wherein the controller is adapted for simultaneous collection of ECG data and oximeter data from a subject.

14. The IRTD of claim 1, wherein the charging coil is positioned on a non-metallic mount.

15. The IRTD of claim 1, wherein the charging coil is positioned on an outer surface the IRTD electrically isolated from a metal case of the IRTD by a non-conductive base.

16. The IRTD of claim 8, wherein the controller is adapted for analysis of ECG signals.

17. The IRTD of claim 16, wherein the analysis results in generation of an alarm related to one or more of arrhythmia, ventricular fibrillation, or pulseless ventricular tachycardia (VT).

18. The IRTD of claim 17, wherein the alarms generated by the IRTD is transmitted to the external device.

19. The IRTD of claim 18, wherein the external device comprises a location function and wherein the alarm generated by the IRTD is transmitted to a remote server by the external device along with the determined location of external device.
